(19) United States
(12) Patent Application Publication
(10) Pub. No.: US 2004/0105833 A1
(43) Pub. Date: Jun. 3, 2004

(54) Cosmetic compositions containing a starch phosphate and a cationic polymer and uses thereof

(76) Inventors: Geraldine Fack, Levallois-Perret (FR); Chrystel Pourille-Grethen, Clichy (FR); Serge Restle, Saint-Prix (FR)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/473,431

(22) PCT Filed: Mar. 29, 2002
(86) PCT No.: PCT/FR02/01113

(30) Foreign Application Priority Data
Mar. 30, 2001 (FR) 01/04387

(51) Int. Cl. A61K 7/06; A61K 7/11

(52) U.S. Cl. 424/70.13; 424/70.16

(57) ABSTRACT

The invention concerns novel cosmetic compositions comprising in a cosmetically acceptable medium at least a starch phosphate and at least a particular non-siliconized cationic polymer. Said association provides a fluid texture to the cosmetic compositions, and said compositions are easily rinsed away. Hair treated with said composition have a soft residue-tree feel. Said compositions are used in particular for washing and/or conditioning keratinous materials such as hair or the skin.

The present invention relates to novel cosmetic compositions comprising, in a cosmetically acceptable medium, at least one particular nonsilicone cationic polymer and at least one starch phosphate.

It is well known that hair which has been sensitized (i.e. damaged and/or made fragile) to various degrees under the action of atmospheric agents or under the action of mechanical or chemical treatments, such as dyeing, bleaching and/or permanent waving, is often difficult to disentangle and to style, and lacks softness.

Cosmetic compositions containing thickening polysaccharides, such as in particular starch or celluloses, have already been proposed for the treatment of keratinous materials, and in particular of hair.

Such compositions exhibit, however, disadvantages such as the problems of rinsability, problems of stability at acidic pH, difficulties of distribution over keratinous materials and inadequate cosmetic properties.

The use of cationic polymers to facilitate the disentanglement of the hair and to impart softness and suppleness to it has already been recommended in compositions for the washing or care of keratinous materials such as the hair. The use of cationic polymers for this purpose exhibits various disadvantages. Because of their high affinity for the hair, some of these polymers become substantially deposited during repeated use, leading to undesirable effects such as a charged, unpleasant feel, a stiffening of the hair, and an interfibre adhesion affecting hair styling.

In summary, it is found that current cosmetic compositions containing cationic polymers are not completely satisfactory.

The Applicant has now discovered that the combination of a starch phosphate with certain cationic polymers makes it possible to overcome these disadvantages.

Thus, following major research studies carried out on the subject, it has now been found by the Applicant that by introducing a starch phosphate into the compositions, in particular hair compositions of the prior art based on certain cationic polymers, it is possible to limit, or even eliminate, the problems mentioned above.

Furthermore, this combination imparts a melting texture on the cosmetic compositions, that is to say that it rapidly disappears in the hair. Hair treated with this composition has a soft feel and no residues.

Moreover, the compositions of the invention, when applied to the skin, in particular in foam bath or shower gel form, provide an improvement in the softness of the skin.

Thus, according to the present invention, novel cosmetic compositions are now provided, comprising, in a cosmetically acceptable medium, at least one starch phosphate and at least one nonsilicone cationic polymer as defined hereinbelow.

Another subject of the invention relates to the use of a starch phosphate in, or for the manufacture of a cosmetic composition comprising a nonsilicone cationic polymer as defined hereinbelow.

The different subjects of the invention will now be presented in detail. All the meanings and definitions of the compounds used in the present invention which are given below are valid for all the subjects of the invention.

For the purposes of the present invention, the expression starch phosphate is understood to mean the products derived from the esterification of a starch with a phosphorus-containing derivative, leading to the formation of at least one ester bond between the phosphorus-containing derivative and at least one hydroxyl group of the starch.

The starches which can be used in the present invention are more particularly macromolecules in the form of polymers consisting of base units which are anhydroglucose units. The number of these units and their assembly make it possible to distinguish between amylose (linear polymer) and amylopectin (branched polymer). The relative proportions of amylose and amylopectin, as well as their degree of polymerization, vary according to the botanical origin of the starches.

The molecules of starches used in the present invention may have cereals or tubers as botanical origin. Thus, the starches are for example chosen from maize, rice, cassava, tapioca, barley, potato, wheat, sorghum and pea starches.

Starches generally exist in the form of a white powder, insoluble in cold water, whose elementary particle size ranges from 3 to 100 microns.

According to the invention, the starches may be optionally C1-C6 hydroxyalkylated or C1-C6 acylated (preferably acetylated). The starches may have also undergone heat treatments.

It is possible to obtain either monostarch phosphates (of the St-O—PO—(OX)₂ type) or distarch phosphates (of the St-O—PO—(OX)—O-St type) or even tristarch phosphates (of the St-O—PO—(O-St)₂ type) or mixtures thereof.

X denotes in particular alkali metals (for example sodium or potassium), alkaline earth metals (for example calcium, magnesium), salts of ammonium hydroxide, salts of amines such as those of monoethanolamine, diethanolamine, triethanolamine, 3-amino-1,2-propanediol, ammonium salts derived from basic amino acids such as lysine, arginine, sarcosine, ornithine and citrulline.

The phosphorus-containing compounds may be, for example, sodium tripolyphosphate, sodium orthophosphate, phosphorus oxychloride or sodium trimetaphosphate.

There will be preferably used distarch phosphates or compounds rich in distarch phosphate, such as the product provided under the references PREJEL VA-70-T AGGL (gelatinized hydroxypropylated cassava distarch phosphate) or PREJEL TK1 (gelatinized cassava distarch phosphate) or PREJEL 200 (gelatinized acetylated cassava distarch phosphate) by the company AVEBE.

The starch phosphate(s) are preferably used in a quantity ranging from 0.01 to 20% by weight relative to the total weight of the composition. More preferably, this quantity ranges from 0.05 to 15% by weight relative to the total weight of the composition, and more preferably still ranging from 0.1 to 10% by weight.

Still more generally, for the purposes of the present invention, the expression “cationic polymer” denotes any polymer containing cationic groups and/or groups ionizable into cationic groups.

The term “nonsilicone” means that the cationic polymer contains no silicon.

The cationic polymers used generally have a number-average molecular mass ranging from 500 to 5×10⁶ approximately, and preferably ranging from 10³ to 3×10⁶ approximately.

The cationic polymers are chosen from

(1) the homopolymers or copolymers derived from acrylic or methacrylic esters or amides and comprising 10 at least one of the units of the following formulae:

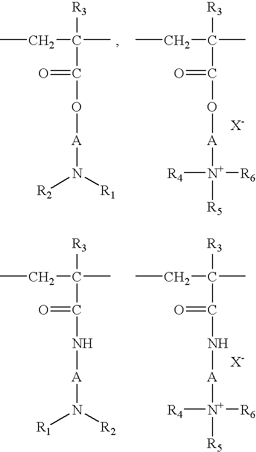

in which:

R₃, which are identical or different, denote a hydrogen atom or a CH₃ radical;

A, which are identical or different, represent a linear or branched alkyl group of 1 to 6 carbon atoms, preferably 2 or 3 carbon atoms or a hydroxyalkyl group of 1 to 4 carbon atoms;

R₄, R₅, R₆, which are identical or different, represent an alkyl group having from 1 to 18 carbon atoms or a benzyl radical and preferably an alkyl group having from 1 to 6 carbon atoms;

R₁ and R₂, which are identical or different, represent hydrogen or an alkyl group having from 1 to 6 carbon atoms and preferably methyl or ethyl;

X denotes an anion derived from an inorganic or organic acid such as a methosulphate anion or a halide such as chloride or bromide.

The copolymers of the family (1) may contain, in addition, one or more units derived from comonomers which may be chosen from the family of acrylamides, methacrylamides, diacetone acrylamides, acrylamides and methacrylamides substituted on the nitrogen with lower (C₁-C₄)alkyls, acrylic or methacrylic acids or esters thereof, vinyllactams such as vinylpyrrolidone or vinylcaprolactam, vinyl esters.

Thus, among these copolymers of the family (1), there may be mentioned:

the copolymers of acrylamide and dimethylamino-ethyl methacrylate quaternized with dimethyl sulphate or with a dimethyl halide such as that sold under the name HERCOFLOC by the company HERCULES,

the copolymers of acrylamide and methacryloyloxy-ethyltrimethylammonium chloride described, for example, in Patent Application EP-A-080976 and sold under the name BINA QUAT P 100 by the company CIBA GEIGY,

the copolymer of acrylamide and methacryloyloxy-ethyltrimethylammonium methosulphate sold under the name RETEN by the company HERCULES,

the vinylpyrrolidone/dialkylaminoalkyl acrylate or methacrylate copolymers, quaternized or otherwise, such as the products sold under the name “GAFQUAT” by the company ISP such as for example “GAFQUAT 734” or “GAFQUAT 755” or alternatively the products called “COPOLYMER 845, 958 and 937”. These polymers are described in detail in French Patents 2,077,143 and 2,393,573,

except for the dimethylaminoethyl methacrylate/vinylcaprolactam/vinylpyrrolidone terpolymers, the vinylpyrrolidone/dimethylaminopropyl methacrylamide copolymers and the vinylpyrrolidone/methacrylamido-propyldimethylamine copolymers

(2) The cationic celluloses. Among the cationic celluloses, there may be mentioned more particularly the cellulose ether derivatives comprising quaternary ammonium groups, the cationic cellulose copolymers or the cellulose derivatives grafted with a water-soluble ammonium monomer.

The cellulose ether derivatives comprising quaternary ammonium groups described in French Patent 1,492,597, and in particular the polymers marketed under the names “JR” (JR 400, JR 125, JR 30M) or “LR” (LR 400, LR 30M) by the company Union Carbide Corporation. These polymers are also defined in the CTFA dictionary as hydroxyethyl cellulose quaternary ammoniums which have reacted with an epoxide substituted by a trimethyl-ammonium group.

Cationic cellulose copolymers or cellulose derivatives grafted with a quaternary ammonium water-soluble monomer, are described especially in U.S. Pat. No. 4,131,576, such as hydroxyalkyl celluloses like hydroxymethyl, hydroxyethyl or hydroxypropyl celluloses grafted especially with a methacryloylethyl-trimethylammonium, methacrylmidopropyltrimethylammonium or dimethyldiallylammonium salt.

The commercialized products corresponding to this definition are more particularly the products sold under the name “Celquat L 200” and “Celquat H 100” by the company National Starch.

(3) Polymers consisting of piperazinyl units and of alkylene or hydroxyalkylene divalent radicals with straight or branched chains, optionally interrupted by oxygen, sulphur or nitrogen atoms or by aromatic or heterocyclic rings, as well as the oxidation and/or quaternization products of these polymers. Such polymers are described especially in French patents 2,162,025 and 2,280,361;

(4) Water-soluble polyaminoamides prepared in particular by polycondensation of an acid compound with a polyamine; these polyaminoamides may be crosslinked with an epihalohydrin, a diepoxide, a dianhydride, an unsaturated dianhydride, a diunsaturated derivative, a bishalohydrin, a bisazetidinium, a bishaloacyldiamine, an alkylbishalide or else with an oligomer resulting from the reaction of a difunctional compound which is reactive towards a bishalohydrin, a bisazetidinium, a bishaloacyldiamine, an alkylbishalide, an epihalohydrin, a diepoxide or a diunsaturated derivative; the crosslinking agent being employed in proportions ranging from 0.025 to 0.35 mol per amine group of the polymaoamide; these polyaminoamides may be alkylated or, if they include one or more tertiary amine functional groups, quaternized. Such polymers are described especially in French Patents 2,252,840 and 2,368,508.

(5) Polyaminoamide derivatives resulting from the condensation of polyalkylenepolyamines with polycarboxylic acids, followed by an alkylation with bifunctional agents. There may be mentioned, for example, the adipic acid-dialkylaminohydroxy-alkyldialkylenetriamine polymers in which the alkyl radical contains from 1 to 4 carbon atoms and preferably denotes methyl, ethyl or propyl. Such polymers are described especially in French Patent 1,583,363.

Among these derivatives there may be mentioned more particularly the adipic acid/dimethyl-aminohydroxypropyl/diethylenetriamine polymers sold under the name “Cartaretine F, F4 or F8” by the company Sandoz.

(6) Polymers obtained by reaction of a polyalkylenepolyamine containing two primary amine groups and at least one secondary amine group with a dicarboxylic acid chosen from diglycolic acid and saturated aliphatic dicarboxylic acids containing from 3 to 8 carbon atoms. The molar ratio of the polyalkylenepolyamine to the dicarboxylic acid ranging from 0.8:1 to 1.4:1; the polyaminoamide resulting therefrom being made to react with epichlorohydrin in a molar ratio of epichlorohydrin relative to the secondary amine group of the polyaminoamide ranging from 0.5:1 to 1.8:1. Such polymers are described especially in U.S. Pat. Nos. 3,227,615 and 2,961,347.

Polymers of this type are marketed in particular under the name “Hercosett 57” by the company Hercules Inc. or else under the name of “PD 170” or “Delsette 101” by the company Hercules in the case of the copolymer of adipic acid/epoxypropyl/diethyl-triamine.

(7) Cyclopolymers of alkyldiallylamine or of dialkyldiallylammonium, such as the homopolymers or copolymers comprising, as main constituent of the chain, units corresponding to the formulae (VI) or (VI′):

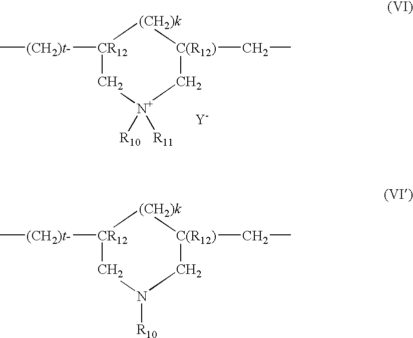

in which formulae k and t are equal to 0 or 1, the sum k+t being equal to 1; R₁₂ denotes a hydrogen atom or a methyl radical; R₁₀ and R₁₁, independently of each other, denote an alkyl group containing from 1 to 22 carbon atoms, a hydroxyalkyl group in which the alkyl group preferably has 1 to 5 carbon atoms, or a lower (C₁-C₄)amidoalkyl group or R₁₀ and R₁₁ may denote, jointly with the nitrogen atom to which they are attached, heterocyclic groups such as piperidinyl or morpholinyl; Y⁻ is an anion such as bromide, chloride, acetate, borate, citrate, tartrate, bisulphate, bisulphite, sulphate or phosphate,

except for the copolymer of diallyl-dimethylammonium chloride and acrylamide in particular marketed under the name “MERQUAT 550”.

These polymers are described especially in French Patent 2,080,759 and in its certificate of addition 2,190,406.

R₁₀ and R₁₁, independently of each other, preferably denote an alkyl group having from 1 to 4 carbon atoms.

Among the polymers defined above there may be mentioned more particularly the homopolymers comprising, as main constituent of the chain, units corresponding to the formulae (VI) or (VI′) such as, in particular, the dimethyldiallylammonium chloride homopolymer sold, for example, under the name “MERQUAT 100” by the company CALGON (and its homologues of low weight-average molecular masses).

(8) The quaternary diammonium polymer containing repeat units corresponding to the formula:

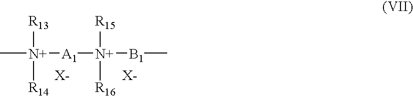

formula (VII) in which:

R₁₃, R₁₄, R₁₅ and R₁₆, which are identical or different, represent aliphatic, alicyclic or arylaliphatic radicals containing from 1 to 20 carbon atoms or lower hydroxyalkyl aliphatic radicals, or else R₁₃, R₁₄, R₁₅ and R₁₆, together or separately, form, with the nitrogen atoms to which they are attached, heterocyclic rings optionally containing a second heteroatom other than nitrogen, or else R₁₃, R₁₄, R₁₅ and R₁₆ represent a linear or branched C₁-C₆ alkyl radical substituted by a nitrile, ester, acyl, amide or —CO—O—R₁₇-D or —CO—NH—R₁₇-D group where R₁₇ is an alkylene and D a quaternary ammonium group;

A₁ and B₁ represent polymethylene groups containing from 2 to 20 carbon atoms which may be linear or branched, saturated or unsaturated and which may contain, bonded to or inserted into the main chain, one or more aromatic rings, or one or more oxygen or sulphur atoms or sulphoxide, sulphone, disulphide, amino, alkylamino, hydroxyl, quaternary ammonium, ureido, amide or ester groups, and

X⁻ denotes an anion derived from an inorganic or organic acid;

A₁, R₁₃ and R₁₅, with the two nitrogen atoms to which they are attached, may form a piperazine ring; in addition if A₁ denotes a saturated or unsaturated, linear or branched alkylene or hydroxyalkylene radical, B₁ may also denote a group (CH₂)ₙ—CO-D-OC—(CH₂)ₙ—

in which D denotes:

a) a glycol residue of formula: —O-Z-O—, where Z denotes a linear or branched hydrocarbon radical or a group corresponding to one of the following formulae:

13 (CH₂—CH₂—O)ₓ—CH₂—CH₂—

—[CH₂—CH(CH₃)—O]_{y}—CH₂—CH(CH₃)—

where x and y denote an integer from 1 to 4, representing a defined and unique degree of polymerization or any number from 1 to 4 representing a mean degree of polymerization;

b) a disecondary diamine residue such as a piperazine derivative;

c) a diprimary diamine residue of formula: —NH—Y—NH—, where Y denotes a linear or branched hydrocarbon radical or else the divalent radical

—CH₂—CH₂—S—S—CH₂—CH₂—;

d) a ureylene group of formula: —NH—CO—NH—;

X⁻ is preferably an anion such as chloride or bromide.

These polymers have a number-average molecular mass which generally ranges from 1000 to 100,000.

Polymers of this type are described especially in French Patents 2,320,330, 2,270,846, 2,316,271, 2,336,434 and 2,413,907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

It is possible to use more particularly the polymers which consist of repeat units corresponding to the formula:

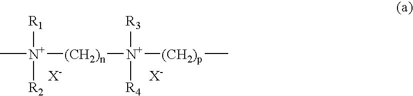

in which R₁, R₂, R₃ and R₄, which are identical or different, denote an alkyl or hydroxyalkyl radical having from 1 to 4 carbon atoms approximately, n and p are integers varying from 2 to 20 approximately and X⁻ is an anion derived from an inorganic or organic acid.

An especially preferred compound of formula (a) is that for which R₁, R₂, R₃ and R₄ represent a methyl radical and n=3, p=6 and X=Cl, called Hexadimethrine chloride according to the INCI nomenclature (CTFA).

(9) Quaternary polyammonium polymers consisting of units of formula (VIII):

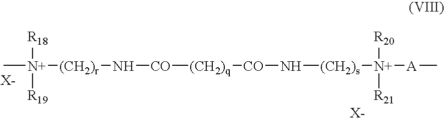

in which formula:

R₁₈, R₁₉, R₂₀ and R₂₁, which are identical or different, represent a hydrogen atom or a methyl, ethyl, propyl, β-hydroxyethyl, β-hydroxypropyl or —CH₂CH₂ (OCH₂CH₂)ₚOH radical,

where p is equal to 0 or to an integer ranging from 1 to 6, provided that R₁₈, R₁₉, R₂₀ and R₂₁ do not simultaneously denote a hydrogen atom,

r and s, which are identical or different, are integers ranging from 1 to 6,

q is equal to 0 or to an integer ranging from 1 to 34,

X⁻denotes an anion such as a halide,

A denotes a radical of a dihalide or preferably represents —CH₂—CH₂—O—CH₂—CH₂—.

Such compounds are described especially in Patent Application EP-A-122 324. Among these there may be mentioned, for example, the products “MIRAPOL® A 15”, “MIRAPOL® AD1”, “MIRAPOL® AZ1” and “MIRAPOL® 175”, sold by the company MIRANOL.

(10) Quaternary vinylpyrrolidone and vinylimidazole polymers such as, for example, the products marketed under the names LUVIQUAT® FC 905, FC 550 and FC 370 by the company B.A.S.F.

(11) Polycondensates of ethoxylated (C₈-C₂₆)alkyl-amines, and of (C₁-C₈)alkylenediamine and, where appropriate, of epichlorohydrin such as, for example, POLYQUART® H 81 sold by COGNIS, referred to under the name of “PEG (15) COCOPOLYAMINE” in the CTFA dictionary.

(12) The crosslinked or uncrosslinked polymers (homopolymer or copolymer) of methacryloyloxy(C₁-C₄ alkyl)tri(C₁-C₄ alkyl)ammonium salts such as the polymers obtained by homopolymerization of dimethylaminoethyl methacrylate quaternized with methyl chloride, or by copolymerization of acrylamide or methacrylamide with dimethylaminoethyl methacrylate quaternized with methyl chloride, the homo- or copolymerization being followed by crosslinking with a compound containing olefinic unsaturation, in particular methylenebisacrylamide. More particularly, it is possible to employ a crosslinked acrylamide/methacryloyloxyethyltrimethylammonium chloride copolymer (20/80 by weight) in the form of a dispersion containing 50% by weight of the said copolymer in mineral oil. This dispersion is marketed under the name of “SALCARE® SC 92” by the company ALLIED COLLOIDS. It is also possible to employ a crosslinked methacryloyloxyethyltrimethylammonium chloride homopolymer containing approximately 50% by weight of the homopolymer in mineral oil or in a liquid ester. These dispersions are marketed under the names of “SALCARE® SC 95” and “SALCARE® SC 96” by the company ALLIED COLLOIDS.

Other cationic polymers that may be employed within the scope of the invention are cationic proteins or hydrolysates of cationic proteins, polyalkylene-imines, in particular polyethyleneimines, polymers containing vinylpyridine or vinylpyridinium units, condensates of polyamines and of epichlorohydrin, quaternary polyureylenes and chitin derivatives.

Among all the cationic polymers capable of being used within the scope of the present invention, it is preferable to use quaternary cellulose ether derivatives such as the products sold under the name “JR 400” by the company UNION CARBIDE CORPORATION, the homopolymers of dimethyldiallylammonium chloride, sold under the name “MERQUAT 100” by the company CALGON, quaternary vinylpyrrolidone and vinylimidazole polymers, the crosslinked or noncrosslinked homopolymers or copolymers of methacryloyloxy(C₁-C₄)alkyl(C₁-C₄)trialkylammonium salts and mixtures thereof.

According to the invention, the cationic polymer(s) may represent from 0.001% to 20% by weight, preferably from 0.01% to 10% by weight, and more particularly from 0.1 to 5% by weight relative to the total weight of the final composition.

According to a particularly preferred embodiment, the compositions according to the invention comprise, in addition, at least one silicone or another agent which is beneficial to the hair, such as in particular the esters of C₁-C₃₀ carboxylic acids and of mono- or polyhydroxylated C₁-C₃₀ alcohols, vegetable, animal, mineral or synthetic oils, waxes, ceramides or pseudoceramides.

The silicones which can be used in accordance with the invention are in particular polyorganosiloxanes which are insoluble in the composition and they may be provided in the form of oils, waxes, resins or gums.

The organopolysiloxanes are defined in greater detail in the book by Walter NOLL “Chemistry and Technology of Silicones” (1968) Academie Press. They may be volatile or nonvolatile.

When they are volatile, the silicones are more particularly chosen from those possessing a boiling point of between 60° C. and 260° C., and more particularly still from:

(i) cyclic silicones comprising from 3 to 7 silicon atoms, and preferably 4 to 5. They are, for example, the octamethylcyclotetrasiloxane marketed in particular under the name “VOLATILE SILICONE 7207” by UNION CARBIDE or “SILBIONE 70045 V 2” by RHONE POULENC, the decamethylcyclopentasiloxane marketed under the name “VOLATILE SILICONE 7158” by UNION CARBIDE, “SILBIONE 70045 V 5” by RHONE POULENC, and mixtures thereof.

There may also be mentioned cyclocopolymers of the dimethylsiloxane/methylalkylsiloxane type, such as the “SILICONE VOLATILE FZ 3109” marketed by the company UNION CARBIDE, having the chemical structure:

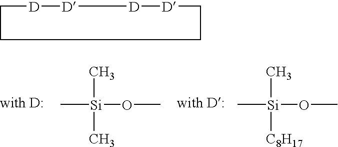

There may also be mentioned mixtures of cyclic silicones with organic compounds derived from silicon, such as the mixture of octamethylcyclotetrasiloxane and tetratrimethylsilylpentaerythritol (50/50) and the mixture of octamethylcyclotetrasiloxane and 1,1′-oxy(2,2,2′,2′,3,3′-hexatrimethylsilyloxy)bisneopentane;

(ii) linear volatile silicones having 2 to 9 silicon atoms and possessing a viscosity of less than or equal to 5×10⁻⁶ m²/s at 25° C. It is for example the decamethyltetrasiloxane marketed in particular under the name “SH 200” by the company TORAY SILICONE. Silicones entering into this class are also described in the article published in Cosmetics and toiletries, Vol. 91, Jan. 76, p. 27-32-TODD & BYERS “Volatile Silicone fluids for cosmetics”.

Nonvolatile silicones and more particularly polyalkylsiloxanes, polyarylsiloxanes, polyalkylaryl-siloxanes, silicone gums and resins, polyorgano-siloxanes modified by organofunctional groups and mixtures thereof are preferably used.

These silicones are more particularly chosen from polyalkylsiloxanes among which there may be mentioned mainly polydimethylsiloxanes with terminal trimethylsilyl groups having a viscosity of 5×10⁻⁶ to 2.5 m²/s at 25° C. and preferably 1×10⁻⁵ to 1 m²/s. The viscosity of the silicones is, for example, measured at 25° C. according to the ASTM 445 Appendix C standard.

Among these polyalkylsiloxanes, there may be mentioned, without limitation, the following commercial products:

SILBIONE oils of the 47 and 70 047 series or MIRASIL oils marketed by RHONE POULENC such as, for example, the 70 047 V 500 000 oil;

oils of the MIRASIL series marketed by the company RHONE POULENC;

oils of the 200 series from the company DOW CORNING such as more particularly DC200 having a viscosity of 60 000 Cst;

VISCASIL oils from GENERAL ELECTRIC and certain oils of the SF series (SF 96, SF 18) from GENERAL ELECTRIC.

There may also be mentioned the polydimethyl-siloxanes with terminal dimethylsilanol groups (Dimethiconol according to the CTFA name), such as the oils of the 48 series from the company RHONE POULENC.

In this class of polyalkylsiloxanes, there may also be mentioned the products marketed under the names “ABIL WAX 9800 and 9801” by the company GOLDSCHMIDT which are poly(C₁-C₂₀)alkylsiloxanes.

The polyalkylarylsiloxanes are particularly chosen from polydimethyl methylphenylsiloxanes, poly-dimethyl diphenylsiloxanes which are linear and/or branched and have a viscosity of 1×10⁻⁵ to 5×10⁻² m²/s at 25° C.

Among these polyalkylarylsiloxanes, there may be mentioned, by way of example, the products marketed under the following names:

SILBIONE oils of the 70 641 series from RHONE POULENC;

oils of the RHODORSIL 70 633 and 763 series from RHONE POULENC;

DOW CORNING 556 COSMETIC GRAD FLUID oil from DOW CORNING;

silicones of the PK series from BAYER such as the product PK20;

silicones of the PN, PH series from BAYER such as the products PN1000 and PH1000;

certain oils of the SF series from GENERAL ELECTRIC such as SF 1023, SF 1154, SF 1250, SF 1265.

The silicone gums which can be used in accordance with the invention are in particular polydiorganosiloxanes having high number-average molecular masses ranging from 200,000 to 1,000,000, used alone or as a mixture in a solvent. This solvent may be chosen from volatile silicones, poly-dimethylsiloxane (PDMS) oils, polyphenylmethylsiloxane (PPMS) oils, isoparaffins, polyisobutylenes, methylene chloride, pentane, dodecane, tridecane or mixtures thereof.

The following products may be more particularly mentioned:

polydimethylsiloxane,

polydimethylsiloxane/methylvinylsiloxane gums,

polydimethylsiloxane/diphenylsiloxane,

polydimethylsiloxane/phenylmethylsiloxane,

polydimethylsiloxane/diphenylsiloxane/methyl-vinylsiloxane.

Products which can be more particularly used in accordance with the invention are mixtures such as:

the mixtures formed from a polydimethyl-siloxane hydroxylated at the chain end (called dimethiconol according to the nomenclature of the CTFA dictionary) and from a cyclic dimethylsiloxane (called cyclomethicone according to the nomenclature of the CTFA dictionary) such as the product Q2 1401 marketed by the company DOW CORNING;

the mixtures formed from a polydimethyl-siloxane gum with a cyclic silicone such as the product SF 1214 Silicone Fluid from the company GENERAL ELECTRIC; this product is a gum SF 30 corresponding to a dimethicone, having a number-average molecular weight of 500 000, solubilized in the oil SF 1202 Silicone Fluid corresponding to decamethylcyclopentasiloxane;

the mixtures of two PDMSs of different viscosities, and more particularly of a PDMS gum and a PDMS oil, such as the product SF 1236 from the company GENERAL ELECTRIC. The product SF 1236 is the mixture of an SE 30 gum defined above having a viscosity of 20 m²/s and an SF 96 oil having a viscosity of 5×10⁻⁶ m²/s. This product preferably comprises 15% of SE 30 gum and 85% of an SF 96 oil.

The organopolysiloxane resins which can be used in accordance with the invention are crosslinked siloxane systems containing the units:

R₂SiO_{2/2}, R₃SiO_{1/2}, RSiO_{3/2} and SiO_{4/2} in which R represents a hydrocarbon group possessing 1 to 16 carbon atoms or a phenyl group. Among these products, those particularly preferred are those in which R denotes a C₁-C₄ lower alkyl, more particularly methyl, radical or a phenyl radical.

There may be mentioned among these resins the product marketed under the name “DOW CORNING 593” or those marketed under the names “SILICONE FLUID SS 4230 and SS 4267” by the company GENERAL ELECTRIC and which are silicones having the dimethyl/trimethylsiloxane structure.

There may also be mentioned the resins of the trimethylsilyloxysilicate type which are marketed in particular under the names X22-4914, X21-5034 and X21-5037 by the company SHIN-ETSU.

The organomodified silicones which can be used in accordance with the invention are silicones as defined above and comprising in their structure one or more organofunctional groups attached via a hydrocarbon group.

Among the organomodified silicones, there may be mentioned the polyorganosiloxanes comprising:

polyethyleneoxy and/or polypropyleneoxy groups optionally comprising C₆-C₂₄ alkyl groups such as the products called dimethicone-copolyol marketed by the company DOW CORNING under the name DC 1248 or the oils SILWETO L 722, L 7500, L 77, L 711 from the company UNION CARBIDE and the (C₁₂)alkyl methicone-copolyol marketed by the company DOW CORNING under the name Q2 5200;

substituted or unsubstituted amine-containing groups such as the products marketed under the name GP 4 Silicone Fluid and GP 7100 by the company GENESEE or the products marketed under the names Q2 8220 and DOW CORNING 929 or 939 by the company DOW CORNING. The substituted amine-containing groups are in particular C₁-C₄ aminoalkyl groups;

thiol groups, such as the products marketed under the names “GP 72 A” and “GP 71” from GENESEE;

alkoxylated groups, such as the product marketed under the name “SILICONE COPOLYMER F-755” by SWS SILICONES and ABIL WAX 2428, 2434 and 2440 by the company GOLDSCHMIDT;

hydroxylated groups, such as the polyorgano-siloxanes with a hydroxyalkyl functional group which are described in French patent application FR-A-85 16334 corresponding to the formula (IX):

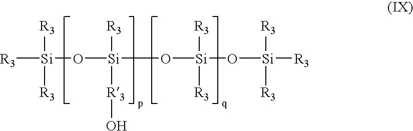

in which the R₃ radicals, which are identical or different, are chosen from methyl and phenyl radicals; at least 60 mol% of the R₃ radicals denoting methyl; the R′₃ radical is a divalent hydrocarbon C₂-C₁₈ alkylene member; p ranges from 1 to 30 inclusive; q ranges from 1 to 150 inclusive;

acyloxyalkyl groups such as, for example, the polyorganosiloxanes described in patent U.S. Pat. No. 4,957,732 and corresponding to the formula (X):

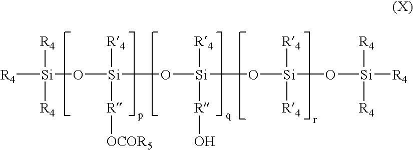

in which:

R₄ denotes a methyl, phenyl, —OCOR₅ or hydroxyl group, it being possible for only one of these R₄ radicals per silicon atom to be OH;

R′₄ denotes methyl or phenyl; at least 60%, as a molar proportion, of all the R₄ and R′₄ radicals denoting methyl;

R₅ denotes a C₈-C₂₀ alkyl or alkenyl;

R″ denotes a linear or branched, divalent hydrocarbon C₂-C₁₈ alkylene radical;

r ranges from 1 to 120 inclusive;

p ranges from 1 to 30;

q is equal to 0 or is less than 0.5 p, p+q ranging from 1 to 30; the polyorganosiloxanes of formula (VI) may contain groups:

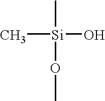

in proportions not exceeding 15% of the sum p+q+r;

anionic groups of the carboxylic type, such as, for example, in the products described in patent EP 186 507 from the company CHISSO CORPORATION, or of the alkylcarboxylic type such as those present in the product X-22-3701E from the company SHIN-ETSU; 2-hydroxyalkylsulphonate; 2-hydroxyalkylthiosulphate such as the products marketed by the company GOLDSCHMIDT under the names “ABIL S201” and “ABIL S255”;

hydroxyacylamino groups, such as the poly-organosiloxanes described in application EP 342 834. There may be mentioned, for example, the product Q2-8413 from the company DOW CORNING.

According to the invention, it is also possible to use silicones comprising a polysiloxane portion and a portion consisting of a nonsilicone organic chain, one of the two portions constituting the principal chain of the polymer, the other being grafted onto the said principal chain. These polymers are for example described in patent applications EP-A-412 704, EP-A-412 707, EP-A-640 105 and WO 95/00578, EP-A-582 152 and WO 93/23009 and U.S. Pat. No. 4,693,935, U.S. Pat. No. 4,728,571 and U.S. Pat. No. 4,972,037. These polymers are preferably anionic or nonionic.

Such polymers are, for example, the polymers which can be obtained by free-radical polymerization starting with a mixture of monomers, consisting of:

a) 50 to 90% by weight of tert-butyl acrylate;

b) 0 to 40% by weight of acrylic acid;

c) 5 to 40% by weight of silicone-containing macromer of formula:

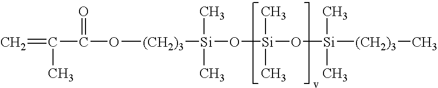

with v being a number ranging from 5 to 700; the percentages by weight being calculated relative to the total weight of the monomers.

Other examples of graft silicone-containing polymers are in particular polydimethylsiloxanes (PDMS) on which are grafted, via a linking member of the thiopropylene type, mixed polymer units of the poly(meth)acrylic acid type and of the polyalkyl (meth)acrylate type, and polydimethylsiloxanes (PDMS) on which are grafted, via a linking member of the thiopropylene type, polymer units of the polyisobutyl (meth)acrylate type.

According to the invention, silicone-containing polyurethanes, in particular those described in patent applications EP 0,751,162 and EP 0,619,111, may also be used.

According to the invention, all the silicones may also be used in the form of emulsions, nano-emulsions or macroemulsions.

The particularly preferred polyorgano-siloxanes in accordance with the invention are:

nonvolatile silicones chosen from the family of polyalkylsiloxanes with trimethylsilyl terminal groups such as oils having a viscosity ranging from 0.2 to 2.5 m²/s at 25° C. such as the oils of the DC200 series from DOW CORNING, in particular that of viscosity 60,000 Cst, of the series SILBIONE 70047 and 47 and more particularly the oil 70 047 V 500 000 marketed by the company RHONE POULENC, the polyalkylsiloxanes with dimethylsilanol terminal groups such as dimethiconol or the polyalkylarylsiloxanes such as the SILBIONE 70641 V 200 oil marketed by the company RHONE POULENC;

the organopolysiloxane resin marketed under the name DOW CORNING 593;

the polysiloxanes with amino groups such as amodimethicones or trimethylsilylamodimethicone.

According to the invention, the esters of C₁-C₃₀ carboxylic acids and C₁-C₃₀ mono- or polyhydroxylated alcohols are chosen in particular from isopropyl myristate, isononyl isononanoate and mixtures thereof.

According to the invention the vegetable oils are chosen in particular from avocado oil, olive oil and mixtures thereof.

According to the invention, the additional silicones or the other additional beneficial agents may represent from 0.001% to 20% by weight, preferably from 0.01% to 10% by weight and more particularly from 0.1 to 5% by weight relative to the total weight of the final composition.

The compositions of the invention advantageously contain, in addition, at least one surfactant which is generally present in a quantity ranging from 0.01% to 50% by weight approximately, preferably ranging from 0.1% to 40% and still more preferably ranging from 0.5% to 30%, relative to the total weight of the composition.

This surfactant may be chosen from anionic, amphoteric, nonionic and cationic surfactants, or mixtures thereof.

The surfactants which are suitable for carrying out the present invention are especially the following:

(i) Anionic Surfactant(s):

Their nature is not of truly critical importance within the context of the present invention.

Thus, by way of example of anionic surfactants that can be employed, by themselves or as mixtures, in the context of the present invention, there may be mentioned especially (nonlimiting list) the salts (in particular alkali metal, especially sodium, salts, ammonium salts, amine salts, amino alcohol salts or magnesium salts) of the following compounds: alkyl sulphates, alkyl ether sulphates, alkylamido ether sulphates, alkylaryl-polyether sulphates, monoglyceride sulphates, alkyl sulphonates, alkyl phosphates, alkylamidesulphonates, alkyl aryl sulphonates, α-olefin-sulphonates, paraffin-sulphonates, alkyl sulphosuccinates, alkyl ether sulphosuccinates, alkylamidesulphosuccinates, alkyl sulphosuccinamates, alkyl sulphoacetates, alkyl ether phosphates, acyl sarcosinates, acyl isethionates and N-acyltaurates, the alkyl or acyl radical of all these different compounds preferably containing from 8 to 24 carbon atoms, and the aryl radical preferably denoting a phenyl or benzyl group. Among the anionic surfactants which are further usable there may also be mentioned the salts of fatty acids such as the salts of oleic, ricinoleic, palmitic and stearic acids, the acids of copra oil or of hydrogenated copra oil, and acyl lactylates in which the acyl radical contains 8 to 20 carbon atoms. It is also possible to employ weakly anionic surfactants, like alkyl-D-galactosideuronic acids and salts thereof, as well as the polyoxyalkylenated carboxylic (C₆-C₂₄)alkyl ether acids, the polyoxyalkylenated carboxylic (C₆-C₂₄)alkylaryl ether acids, the polyoxyalkylenated carboxylic (C₆-C₂₄)alkyl amidoether acids and their salts, in particular those containing from 2 to 50 ethylene oxide groups and mixtures thereof.

Among the anionic surfactants, the use of the salts of alkyl sulphates and of alkyl ether sulphates and mixtures thereof is preferred according to the invention.

(ii) Nonionic Surfactant(s):

The nonionic surfactants themselves are also compounds which are well known per se (in this respect see especially the “Handbook of Surfactants” by M. R. Porter, published by Blackie & Son (Glasgow and London), 1991, pp. 116-178) and, in the context of the present invention, their nature is not of critical importance. They can thus be chosen especially from (nonlimiting list) alcohols, alpha-diols, alkylphenols or polyethoxylated, polypropoxylated or polyglycerolated fatty acids which have a fatty chain containing, for example, 8 to 18 carbon atoms, it being possible for the number of ethylene oxide or propylene oxide groups to range especially from 2 to 50 and it being possible for the number of glycerol groups to range especially from 2 to 30. The copolymers of ethylene oxide and propylene oxide and the condensates of ethylene oxide and propylene oxide with fatty alcohols may also be mentioned; the polyethoxylated fatty amides preferably containing from 2 to 30 mol of ethylene oxide, the polyglycerolated fatty amides on average containing 1 to 5 glycerol groups and in particular 1.5 to 4, the polyethoxylated fatty amines preferably containing 2 to 30 mol of ethylene oxide, the oxyethylenated fatty acid esters of sorbitan containing from 2 to 30 mol of ethylene oxide, the fatty acid esters of sucrose, the fatty acid esters of polyethylene glycol, alkylpolyglycosides, the N-alkylglucamine derivatives, amine oxides such as the oxides of (C₁₀-C₁₄) alkylamines or the N-acylaminopropylmorpholine oxides. It will be noted that alkylpolyglycosides constitute nonionic surfactants which are particularly well suited within the context of the present invention.

(iii) Amphoteric Surfactant(s):

The amphoteric surfactants, the nature of which is not of critical importance in the context of the present invention, may be especially (nonlimiting list) derivatives of aliphatic secondary or tertiary amines in which the aliphatic radical is a linear or branched chain containing 8 to 22 carbon atoms and containing at least one water-solubilizing anionic group (for example carboxylate, sulphonate, sulphate, phosphate or phosphonate); (C₈-C₂₀)alkylbetaines, sulphobetaines, (C₈-C₂₀)alkylamido(C₁-C₆)alkylbetaines or (C₈-C₂₀)alkylamido(C₁-C₆)alkylsulphobetaines may further be mentioned.

Among the amine derivatives there may be mentioned the products sold under the name MIRANOL, as described in U.S. Pat. No. 2,528,378 and U.S. Pat. No. 2,781,354 and of structures:

R₂—CONHCH₂CH₂—N(R₃) (R₄) (CH₂COO—) (2)

in which: R₂ denotes an alkyl radical derived from an acid R₂—COOH present in hydrolysed copra oil, a heptyl, nonyl or undecyl radical, R₃ denotes a beta-hydroxyethyl group and R₄ a carboxymethyl group; and

R₅—CONHCH₂CH₂—N (B) (C) (3)

in which:

B represents —CH₂CH₂OX′, C represents —(CH₂)_{z}—Y′, with z=1 or 2,

X′ denotes the —CH₂CH₂—COOH group or a hydrogen atom,

Y′ denotes —COOH or the radical —CH₂—CHOH—SO₃H,

R₅ denotes an alkyl radical of an acid R₉—COOH present in copra oil or in hydrolysed linseed oil, an alkyl radical, especially C₇, C₉, C₁₁ or C₁₃, a C₁₇ alkyl radical and its iso form or an unsaturated radical C₁₇.

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphodipropionate, Disodium Caprylamphodipropionate, Disodium Capryloamphodipropionate, Lauroamphodipropionic acid, Cocoamphodipropionic acid.

By way of example, there may be mentioned the cocoamphodiacetate marketed under the trade name MIRANOL C2M concentrate by the company RHONE POULENC.

(iv) The Cationic Surfactants May be Chosen From:

A) the quaternary ammonium salts of the following general formula (IV):

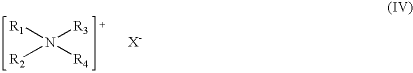

in which X is an anion chosen from the group comprising halides (chloride, bromide or iodide) or (C₂-C₆)alkyl sulphates, more particularly methyl sulphate, phosphates, alkyl or alkylaryl sulphonates, anions derived from organic acid such as acetate or lactate, and

a) the radicals R₁ to R₃, which may be identical or different, represent a linear or branched aliphatic radical comprising from 1 to 4 carbon atoms, or an aromatic radical such as alkyl or alkylaryl. The aliphatic radicals may comprise heteroatoms such as in particular oxygen, nitrogen, sulphur, halogens. The aliphatic radicals are, for example, chosen from alkyl, alkoxy and alkylamide radicals,

R₄ denotes a linear or branched alkyl radical comprising from 16 to 30 carbon atoms.

Preferably, the cationic surfactant is a salt (for example chloride) of behenyltrimethylammonium.

b) The radicals R₁ and R₂, which may be identical or different, represent a linear or branched aliphatic radical comprising from 1 to 4 carbon atoms, or an aromatic radical such as aryl or alkylaryl. The aliphatic radicals may comprise heteroatoms such as, in particular oxygen, nitrogen, sulphur, halogens. The aliphatic radicals are, for example, chosen from alkyl, alkoxy, alkylamide and hydroxyalkyl radicals comprising about from 1 to 4 carbon atoms;

R₃ and R₄, which may be identical or different, denote a linear or branched alkyl radical comprising from 12 to 30 carbon atoms, the said radical comprising at least one ester or amide functional group,

R₃ and R₄ are in particular chosen from the radicals (C₁₂-C₂₂)alkylamido(C₂-C₆)alkyl, (C₁₂-C₂₂)alkyl acetate.

Preferably, the cationic surfactant is a salt (for example chloride) of stearamidopropyldimethyl-(myristylacetate)ammonium,

B)—the quaternary ammonium salts of imidazolinium, such as for example that of the following formula (V):

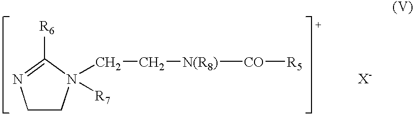

in which R₅ represents an alkenyl or alkyl radical comprising from 8 to 30 carbon atoms, for example derived from tallow fatty acids, R₆ represents a hydrogen atom, a C₁-C₄ alkyl radical or an alkenyl or alkyl radical comprising from 8 to 30 carbon atoms, R₇ represents a C₁-C₄ alkyl radical, R₈ represents a hydrogen atom, a C₁-C₄ alkyl radical, X is an anion chosen from the group comprising halides, phosphates, acetates, lactates, alkyl sulphates, alkyl or alkylaryl sulphonates. Preferably, R₅ and R₆ denote a mixture of alkenyl or alkyl radicals comprising from 12 to 21 carbon atoms, for example derived from tallow fatty acids, R₇ denotes methyl, R₈ denotes hydrogen. Such a product is, for example, Quaternium-27 (CTFA 1997) or Quaternium-83 (CTFA 1997) marketed under the names “REWOQUAT” W 75, W90, W75PG, W75HPG by the company WITCO,

C)—the quaternary diammonium salts of formula (VI):

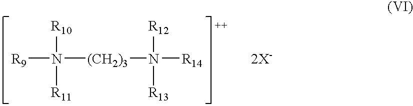

in which R₉ denotes an aliphatic radical comprising about from 16 to 30 carbon atoms, R₁₀, R₁₁, R₁₂, R₁₃, R₁₄, which are identical or different, are chosen from hydrogen or an alkyl radical comprising from 1 to 4 carbon atoms, and X is an anion chosen from the group comprising halides, acetates, phosphates, nitrates and methyl sulphates. Such quaternary diammonium salts optionally comprise propane tallow diammonium dichloride,

D)—the quaternary ammonium salts containing at least one ester functional group of the following formula (VII):

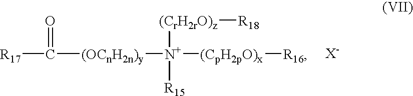

in which:

R₁₅ is chosen from C₁-C₆ alkyl radicals and C₁-C₆ hydroxyalkyl or dihydroxyalkyl radicals;

R₁₆ is chosen from:

the radical

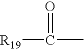

the saturated or unsaturated, linear or branched C₁-C₂₂ hydrocarbon radicals R₂₀,

a hydrogen atom,

R₁₈ is chosen from:

the radical

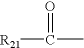

the saturated or unsaturated, linear or branched C₁-C₆ hydrocarbon radicals R₂₂,

a hydrogen atom,

R₁₇, R₁₉ and R₂₁, which are identical or different, are chosen from saturated or unsaturated, linear or branched C₇-C₂₁ hydrocarbon radicals;

n, p and r, which are identical or different, are integers ranging from 2 to 6;

y is an integer ranging from 1 to 10;

x and z, which are identical or different, are integers ranging from 0 to 10;

X⁻ is a simple or complex, organic or inorganic anion; provided that x+y+z is equal to 1 to 15, that when x is equal to 0, then R₁₆ denotes R₂₀ and that when z is equal to 0 then R₁₈ denotes R₂₂.

There are more particularly used the ammonium salts of formula (VII) in which:

R₁₅ denotes a methyl or ethyl radical;

x and y are equal to 1;

z is equal to 0 or 1;

n, p and r are equal to 2;

R₁₆ is chosen from:

the radical

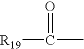

the methyl, ethyl or C₁₄-C₂₂ hydrocarbon radicals,

a hydrogen atom;

R₁₇, R₁₉ and R₂₁, which are identical or different, are chosen from saturated or unsaturated, linear or branched C₇-C₂₁ hydrocarbon radicals;

R₁₈ is chosen from:

the radical

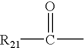

a hydrogen atom.

Such compounds are for example, marketed under the names DEHYQUART by the company HENKEL, STEPANQUAT by the company STEPAN, NOXAMIUM by the company CECA, REWOQUAT WE 18 by the company REWO-WITCO.

Among the quaternary ammonium salts, there is preferred behenyltrimethylammonium salts (for example chloride), or stearamidopropyldimethyl(myristyl-acetate)ammonium salts (for example chloride) marketed under the name “CERAPHYL 70” by the company VAN DYK, palmitylamidopropyltrimethylammonium salts (for example chloride), Quaternium-27 or Quaternium-83 marketed by the company WITCO.

In the compositions according to the invention, there may be used mixture of surfactants and in particular mixtures of anionic surfactants, mixtures of anionic surfactants and of amphoteric, cationic or nonionic surfactants, mixtures of cationic surfactants with nonionic or amphoteric surfactants. A particularly preferred mixture is a mixture consisting of at least one anionic surfactant and at least one amphoteric surfactant.

A composition of the invention may also contain at least one additive chosen from thickeners, perfumes, pearlescent agents, preservatives, sunscreens which are silicone-based or otherwise, vitamins, provitamins, anionic or nonionic polymers, noncationic proteins, noncationic protein hydrolysates, 18-methyl-eicosanoic acid, hydroxy acids, vitamins, provitamins such as panthenol, and any other additive conventionally used in the cosmetic field which does not affect the properties of the compositions according to the invention.

These additives are optionally present in the composition according to the invention in proportions which may range from 0.001 to 20% by weight relative to the total weight of the composition. The precise quantity of each additive is easily determined by persons skilled in the art depending on its nature and its function.

According to one particularly preferred embodiment, the compositions according to the invention comprise a starch phosphate, at least one cationic polymer as defined above, at least one cationic surfactant and at least one silicone.

The compositions in accordance with the invention may be more particularly used for washing or treating keratinous materials such as the hair, the skin, the eyelashes, the eyebrows, the nails, the lips, the scalp and more particularly the hair.

The compositions according to the invention are detergent compositions such as shampoos, shower gels and foam baths. In this embodiment of the invention, the compositions comprise at least one, generally aqueous, washing base.

The surfactant(s) constituting the washing base may equally well be chosen, alone or as mixtures, from anionic, amphoteric, nonionic and cationic surfactants as defined above. The washing base contains at least one detergent surfactant.

An anionic surfactant is preferably used which is chosen from sodium, triethanolamine or ammonium (C₁₂-C₁₄) alkyl sulphates, oxyethylenated sodium, triethanolamine or ammonium (C₁₂-C₁₄)alkyl ether sulphates containing 2.2 mol of ethylene oxide, sodium cocoyl isethionate and sodium (C₁₄-C₁₆)alphaolefin sulphonate and their mixtures with:

either an amphoteric surfactant such as the amine derivatives called disodium cocoamphodipropionate or sodium cocoamphopropionate marketed in particular by the company RHONE POULENC under the trade name “MIRANOL C2M CONC” in aqueous solution at 38% of active material and under the name MIRANOL C32;

or an amphoteric surfactant of the zwitterionic type such as the alkylbetaines in particular cocobetaine marketed under the name “DEHYTON AB 30” in aqueous solution at 32% AM by the company HENKEL.

The quantity and the quality of the surfactants of the washing base are those sufficient to confer on the final composition a satisfactory foaming and/or detergent power.

Thus, according to the invention, the surfactants may represent from 4% to 50% by weight, preferably from 6% to 35% by weight, and still more preferably from 8% to 25% by weight, of the total weight of the final composition.

The subject of the invention is also a method of treating keratinous materials such as the skin or the hair, characterized in that it consists in applying to the keratinous materials a cosmetic composition as defined above, and then in optionally rinsing with water.

Thus, this method according to the invention allows the retention of the hairstyle, the treatment, the care or the washing of or the removal of make-up from the skin, the hair or any other keratinous material.

The compositions of the invention may also be provided in the form of an after-shampoo to be rinsed off or otherwise, of compositions for permanent waving, for hair straightening, for dyeing or bleaching, or alternatively in the form of rinse-off compositions to be applied before or after dyeing, bleaching, permanent waving or hair straightening or alternatively between the two stages of a permanent waving or a hair straightening treatment.

When the composition is provided in the form of an optional rinse-off after-shampoo, it advantageously contains at least one cationic surfactant, its concentration generally ranging from 0.1 to 10% by weight, and preferably from 0.5 to 5% by weight relative to the total weight of the composition.

The compositions of the invention may also be provided in the form of washing compositions for the skin, and in particular in the form of bath or shower solutions or gels or of make-up removing products.

The compositions according to the invention may also be provided in the form of aqueous or aqueous-alcoholic lotions for skin and/or hair care.

The cosmetic compositions according to the invention may be provided in the form of a gel, milk, cream, emulsion, thickened lotion or foam and may be used for the skin, nails, eyelashes, lips and more particularly the hair.

The compositions may be packaged in various forms, in particular in vaporizers, pump dispensers or in aerosol containers in order to allow application of the composition in vaporized form or in foam form. Such forms of packaging are advisable, for example, when it is desired to obtain a spray, a lacquer or a foam for treating the hair.

In the text which follows or in the preceding text, the percentages expressed are by weight.

The invention will now be illustrated more fully with the aid of the following examples which should not be considered as limiting it to the embodiments described.

In the examples, AS means active substance.

### EXAMPLE 1

An after-shampoo in accordance with the invention was prepared which has the following composition:

| | | |
|---|---|---|
| Distarch phosphate (PREJEL VA-70-T from AGGL) | 2.5 | g AS |
| Behenyltrimethylammonium chloride | 1.8 | g AS |
| Homopolymer of ethyltrimethylammonium | 1.5 | g AS |
| methacrylate chloride (SYNTHALEN CR from 3V) | | |
| Amodimethicon (BELSIL ADM 6057 E from | 1.7 | g AS |
| WACKER) | | |
| Water qsp | 100 | g |

The composition has a gel texture and is highly melting on application to wet hair. Its rinsability is good. The wet hair is not charged and it is easy to shape the hair.

### EXAMPLE 2

An after-shampoo in accordance with the invention was prepared which has the following composition:

| | | |
|---|---|---|
| Distarch phosphate (PREJEL VA-70-T from | 5 | g AS |
| AGGL) | | |
| Crosslinked homopolymer of ethyltrimethyl- | 1 | g AS |
| ammonium methacrylate chloride (SALCARE SC 95 | | |
| from CIBA) | | |
| Crosslinked divinyldimethicone/dimethicone | 5 | g AS |
| as a cationic emulsion at 65% of AS (DC2-1997 | | |
| from DOW CORNING) | | |
| Water qsp | 100 | g |

The treated hair has the same properties as that treated with the composition of Example 1.

### EXAMPLE 3

An after-shampoo in accordance with the invention was prepared which has the following composition:

Distarch phosphate (PREJEL VA-70-T from

| | | |
|---|---|---|
| AGGL) | 8 | g AS |
| Palmitylamidopropyltrimethylammonium | 4 | g AS |
| chloride at 60% in propyleneglycol (VARISOFT | | |
| PATC from GOLDSCHMIDT) | | |
| Crosslinked homopolymer of ethyltrimethyl- | 0.1 | g AS |
| ammonium methacrylate chloride (SALCARE SC 95 | | |
| from CIBA) | | |
| Isopropyl myristate | 2 | g |
| Water qsp | 100 | g |

The treated hair has the same properties as that treated with the composition of Example 1.

### EXAMPLE 4

An after-shampoo in accordance with the invention was prepared which has the following composition:

| | | |
|---|---|---|
| Distarch phosphate (PREJEL VA-70-T from | 6 | g AS |
| AGGL) | | |
| Behenyltrimethylammonium chloride | 2.5 | g AS |
| Copolymer of methacrylamide and of | 0.8 | g AS |
| methacrylateethyltrimethylammonium chloride | | |
| (Polyquaternium-15) (ROHAGIT KF 720 from ROHM) | | |
| Crosslinked divinyldimethicone/dimethicone | 1.5 | g AS |
| as a cationic emulsion (DC2-1997 from DOW | | |
| CORNING) | | |
| Water qsp | 100 | g |

The treated hair has the same properties as that treated with the composition of Example 1.

### EXAMPLE 5

An after-shampoo in accordance with the invention was prepared which has the following composition:

| | | |
|---|---|---|
| Distarch phosphate (PREJEL VA-70-T from | 7 | g AS |
| AGGL) | | |
| Sodium lauryl ether sulphate containing | 1 | g AS |
| 2.2 mol of ethylene oxide | | |
| Polyquaternium-10 (JR 400 from AMERCHOL) | 1.2 | g AS |
| Avocado oil | 3 | g |
| Water qsp | 100 | g |

The treated hair has the same properties as that treated with the composition of Example 1.

1. Cosmetic composition, characterized in that it comprises, in a cosmetically acceptable medium, at least one starch phosphate and at least one nonsilicone cationic polymer chosen from:
(1) homopolymers or copolymers derived from acrylic or methacrylic esters or amides and comprising at least one of the units of the following formulae: 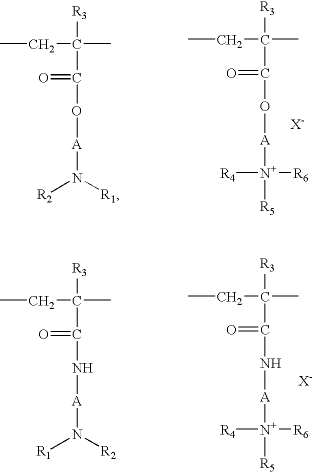
in which:
R₃, which are identical or different, denote a hydrogen atom or a CH₃ radical;
A, which are identical or different, represent a linear or branched alkyl group of 1 to 6 carbon atoms or a hydroxyalkyl group of 1 to 4 carbon atoms;
R₄, R₅, R₆, which are identical or different, represent an alkyl group having from 1 to 18 carbon atoms or a benzyl radical;
R₁ and R₂, which are identical or different, represent hydrogen or an alkyl group having from 1 to 6 carbon atoms;
X denotes an anion derived from an inorganic or organic acid,
(2) cationic celluloses,
(3) polymers consisting of piperazinyl units and of alkylene or hydroxyalkylene divalent radicals with straight or branched chains, optionally interrupted by oxygen, sulphur or nitrogen atoms or by aromatic or heterocyclic rings, as well as the oxidation and/or quaternization products of these polymers,
(4) water-soluble polyaminoamides prepared in particular by polycondensation of an acid compound with a polyamine; these polyaminoamides may be crosslinked with an epihalohydrin, a diepoxide, a dianhydride, an unsaturated dianhydride, a diunsaturated derivative, a bishalohydrin, a bisazetidinium, a bishaloacyldiamine, an alkylbishalide or else with an oligomer resulting from the reaction of a difunctional compound which is reactive towards a bishalohydrin, a bisazetidinium, a bishaloacyldiamine, an alkylbishalide, an epihalohydrin, a diepoxide or a diunsaturated derivative; the crosslinking agent being employed in proportions ranging from 0.025 to 0.35 mol per amine group of the polymaoamide; these polyaminoamides may be alkylated or, if they include one or more tertiary amine functional groups, quaternized,
(4) polyaminoamide derivatives resulting from the condensation of polyalkylenepolyamines with polycarboxylic acids, followed by an alkylation with bifunctional agents,
(6) polymers obtained by reaction of a polyalkylenepolyamine containing two primary amine groups and at least one secondary amine group with a dicarboxylic acid chosen from diglycolic acid and saturated aliphatic dicarboxylic acids containing from 3 to 8 carbon atoms,
(7) cyclopolymers of alkyldiallylamine or of dialkyldiallylammonium, except for the copolymer of diallyldimethylammonium chloride and acrylamide,
(8) quaternary diammonium polymers comprising repeat units corresponding to the formula: 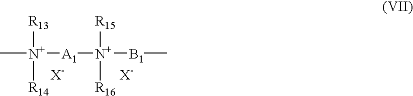
formula (VII) in which:
R₁₃, R₁₄, R₁₅ and R₁₆, which are identical or different, represent aliphatic, alicyclic or arylaliphatic radicals containing from 1 to 20 carbon atoms or lower hydroxyalkyl aliphatic radicals, or else R₁₃, R₁₄, R₁₅ and R₁₆, together or separately, form, with the nitrogen atoms to which they are attached, heterocyclic rings optionally containing a second heteroatom other than nitrogen, or else R₁₃, R₁₄, R₁₅ and R₁₆ represent a linear or branched C₁-C₆ alkyl radical substituted by a nitrile, ester, acyl, amide or —CO—O—R₁₇-D or —CO—NH—R₁₇-D group where R₁₇ is an alkylene and D a quaternary ammonium group;
A₁ and B₁ represent polymethylene groups containing from 2 to 20 carbon atoms which may be linear or branched, saturated or unsaturated and which may contain, bonded to or inserted into the main chain, one or more aromatic rings, or one or more oxygen or sulphur atoms or sulphoxide, sulphone, disulphide, amino, alkylamino, hydroxyl, quaternary ammonium, ureido, amide or ester groups, and
X⁻ denotes an anion derived from an inorganic or organic acid;
A₁, R₁₃ and R₁₅, with the two nitrogen atoms to which they are attached, may form a piperazine ring; in addition if A₁ denotes a saturated or unsaturated, linear or branched alkylene or hydroxyalkylene radical, B₁ may also denote a group (CH₂)ₙ—CO-D-OC—(CH₂)ₙ—
in which D denotes:
a) a glycol residue of formula: —O-Z-O—, where Z denotes a linear or branched hydrocarbon radical or a group corresponding to one of the following formulae:
—(CH₂—CH₂—O)ₓ—CH₂—CH₂—
—[CH₂—CH(CH₃)—O]_{y}—CH₂—CH(CH₃)—
where x and y denote an integer from 1 to 4, representing a defined and unique degree of polymerization or any number from 1 to 4 representing a mean degree of polymerization;
b) a disecondary diamine residue such as a piperazine derivative;
c) a diprimary diamine residue of formula: —NH—Y—NH—, where Y denotes a linear or branched hydrocarbon radical or else the divalent radical
—CH₂—CH₂—S—S—CH₂—CH₂—;
d) a ureylene group of formula: —NH—CO—NH—;
X⁻ is preferably an anion,
(9) quaternary polyammonium polymers consisting of units of formula (VIII): 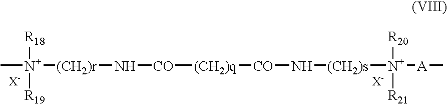
in which formula:
R₁₈, R₁₉, R₂₀ and R₂₁, which are identical or different, represent a hydrogen atom or a methyl, ethyl, propyl, β-hydroxyethyl, β-hydroxypropyl or —CH₂CH₂(OCH₂CH₂)ₚOH radical,
where p is equal to 0 or to an integer ranging from 1 and 6, provided that R₁₈, R₁₉, R₂₀ and R₂₁ do not simultaneously represent a hydrogen atom,
r and s, which are identical or different, are integers ranging from 1 and 6,
q is equal to 0 or to an integer ranging from 1 and 34,
X denotes a halogen atom,
A denotes a radical of a dihalide or preferably represents —CH₂—CH₂—O—CH₂—CH₂—,
(10) quaternary vinylpyrrolidone and vinylimidazole polymers,
(11) polycondensates of ethoxylated (C₈-C₂₆)-alkylamines, of (C₁-C₈)alkylenediamines and, where appropriate, of epichlorohydrin,
(12) crosslinked or noncrosslinked polymers of methacryloyloxy(C₁-C₄ alkyl)tri(C₁-C₄ alkyl)ammonium salts,
(13) polyalkyleneimines, in particular polyethyleneimines, polymers containing vinylpyridine or vinylpyridinium units, condensates of polyamines and epichlorohydrin, quaternary polyureylenes, and chitin derivatives.

2. Composition according to claim 1, characterized in that the starch phosphate is an ester of phosphorus-containing compounds and at least one starch.

3. Composition according to the preceding claim, characterized in that the phosphorus-containing compound is chosen from sodium tripolyphosphate, sodium orthophosphate, phosphorus oxychloride and sodium trimetaphosphate.

4. Composition according to any one of claims 1 to 3, characterized in that the starches are chosen from maize, rice, cassava, tapioca, barley, potato, wheat, sorghum and pea starches.

5. Composition according to any one of claims 1 to 4, characterized in that the starch is C₁-C₆ hydroxyalkylated or C₁-C₆ acylated.

6. Composition according to any one of claims 1 to 5, characterized in that the starch phosphate is chosen from monostarch phosphates, distarch phosphates, tristarch phosphates and mixtures thereof.

7. Composition according to claim 6, characterized in that the starch phosphate is chosen from distarch phosphates.

8. Composition according to any one of claims 1 to 5, characterized in that the starch phosphate is chosen from gelatinized hydroxypropylated cassava distarch phosphates, gelatinized acetylated cassava distarch phosphates and gelatinized cassava distarch phosphates.

9. Composition according to any one of claims 1 to 8, characterized in that the said cationic polymer is chosen from cationic cyclopolymers, cationic celluloses, quaternary vinylpyrrolidone and vinylimidazole polymers, crosslinked or noncrosslinked homopolymers or copolymers of methacryloyloxy(C₁-C₄ alkyl)tri(C₁-C₄ alkyl)ammonium salts and mixtures thereof.

10. Composition according to claim 9, characterized in that the said cyclopolymer is chosen from homopolymers of diallyldimethylammonium chloride.

11. Composition according to claim 9, characterized in that the said cationic celluloses are chosen from hydroxyethylcelluloses which have reacted with an epoxide substituted with a trimethylammonium group.

12. Composition according to any one of claims 1 to 11, characterized in that it comprises, in addition, at least one silicone.

13. Composition according to claim 12, characterized in that the silicones are chosen from polyorganosiloxanes insoluble in the composition.

14. Composition according to either of claims 12 and 13, characterized in that the silicones are nonvolatile polyorganosiloxanes chosen from polyalkylsiloxanes, polyarylsiloxanes, polyalkylarylsiloxanes, silicone gums and resins, polyorganosiloxanes modified by organofunctional groups, and mixtures thereof.

15. Composition according to claim 14, characterized in that:
(a) the polyalkylsiloxanes are chosen from:
polydimethylsiloxanes with trimethylsilyl terminal groups;
polydimethylsiloxanes with dimethylsilanol terminal groups;
poly(C₁-C₂₀)alkylsiloxanes;
(b) the polyalkylarylsiloxanes are chosen from:
polydimethylmethylphenylsiloxanes, polydimethyl-diphenylsiloxanes which are linear and/or branched and have a viscosity ranging from 1×10⁻⁵ and 5×10⁻² m²/s at 25° C.; diphenylsiloxanes (c) the silicone gums are chosen from polydiorgano-siloxanes having number-average molecular masses ranging from 200,000 and 1,000,000, used alone or in the form of a mixture in a solvent;
(d) the resins are chosen from the resins consisting of units: R₃SiO_{1/2}, R₂SiO_{2/2}, RSiO_{3/2}, SiO_{4/2} in which R represents a hydrocarbon group having from 1 to 16 carbon atoms or a phenyl group;
(e) the organomodified silicones are chosen from silicones comprising in their structure one or more organofunctional groups attached via a hydrocarbon radical.

16. Composition according to any one of claims 12 to 15, characterized in that the silicones are chosen from polyalkylsiloxanes with trimethylsilyl terminal groups, polyalkylsiloxanes with dimethylsilanol terminal groups, polyalkyl-arylsiloxanes, mixtures of two PDMSs consisting of a gum and an oil with different viscosities, mixtures of cyclic silicones and organosiloxanes, organopolysiloxane resins, and polysiloxanes containing amino groups.

17. Composition according to claim 16, characterized in that the polysiloxanes containg amino groups are chosen from amodimethicones or tromethylsilylamodimethicones.

18. Composition according to any one of claims 1 to 17, characterized in that it comprises, in addition, at least one beneficial agent chosen from esters of C₁-C₃₀ carboxylic acids and C₁-C₃₀ mono- or polyhydroxylated alcohols, vegetable, animal, mineral or synthetic oils, waxes, ceramides and pseudoceramides.

19. Composition according to claim 18, characterized in that the esters of C₁-C₃₀ carboxylic acids and C₁-C₃₀ mono- or polyhydroxylated alcohols are chosen from isopropyl myristate, isononyl isononanoate and mixtures thereof.

20. Composition according to claim 18, characterized in that the vegetable oils are chosen from avocado oil, olive oil and mixtures thereof.

21. Composition according to any one of the preceding claims, characterized in that the starch phosphate is present at a concentration ranging from 0.01% to 20% by weight relative to the total weight of the composition, preferably from 0.05% to 15% by weight.

22. Composition according to any one of the preceding claims, characterized in that the cationic polymer is present at a concentration ranging from 0.001% to 20% by weight relative to the total weight of the composition, preferably from 0.01% to 10% by weight.

23. Composition according to any one of claims 12 to 22, characterized in that the said silicone is present at a concentration ranging from 0.001% to 20% by weight relative to the total weight of the composition, preferably from 0.01% to 10% by weight.

24. Composition according to any one of claims 17 to 23, characterized in that the agent beneficial to the hair is present at a concentration ranging from 0.001% to 20% by weight relative to the total weight of the composition, preferably from 0.01% to 10% by weight.

25. Composition according to any one of the preceding claims, characterized in that it comprises, in addition, at least one surfactant chosen from anionic, nonionic, amphoteric and cationic surfactants, and mixtures thereof.

26. Composition according to the preceding claim, characterized in that the cationic surfactants are chosen from behenyltrimethylammonium salts, stearamidopropyldimethyl(myristylacetate)ammonium salts, palmitylamidopropyltrimethylammonium salts, Quaternium-27 or Quaternium-83.

27. Composition according to either of claims 25 and 26, characterized in that the surfactant(s) are present at a concentration ranging from 0.01% to 50% by weight, preferably ranging from 0.1% to 40% by weight, and more preferably still ranging from 0.5% to 30% by weight, relative to the total weight of the composition.

28. Composition according to any one of claims 1 to 27, characterized in that it additionally comprises at least one cationic surfactant and at least one silicone.

29. Composition according to any one of the preceding claims, characterized in that it is provided in the form of a shampoo, an after-shampoo, a composition for permanent waving, straightening, dyeing or bleaching the hair, a rinse-off composition to be applied between the two stages of a permanent waving or hair straightening, or a washing composition for the body.

30. Composion according to any one of the preceding claims, characterized in that it is in the form of an after-shampoo, in particular of an after-shampoo to be rinsed off.

31. Use of a composition as defined in any one of the preceding claims, for the washing or for the care of keratinous materials.

32. Method for treating keratinous materials, such as the hair, characterized in that it consists in applying to the said materials a cosmetic composition according to one of claims 1 to 30, and then in optionally rinsing with water.

33. Use of a starch phosphate as defined in one of claims 1 to 8 in or for the manufacture of a cosmetic composition comprising a cationic polymer as defined in any one of claims 1 and 9 to 11.
